(19) United States
(12) Patent Application Publication
(10) Pub. No.: US 2024/0122998 A1
(43) Pub. Date: Apr. 18, 2024

(54) COMPOSITION AND KIT FOR ALLEVIATING SYMPTOMS OF RESPIRATORY ALLERGY

(71) Applicant: SOCIETE DES PRODUITS NESTLE S.A., Vevey (CH)

(72) Inventors: CARINE BLANCHARD, Le Mont-sur-Lausanne (CH); FRANCINE LENFANT, Lausanne (CH); ULRIKE REYMONDIN, Pully (CH); GAELLE MARIE LAURE SCHLUP-OLLIVIER, Lausanne (CH); JOYDEEP RAY, Gurugram - Haryana (IN); SEBASTIEN HOLVOET, Montpreveyres (CH); CHRISTOPHE BARDE, Corseaux (CH)

(21) Appl. No.: 18/546,521

(22) PCT Filed: Feb. 16, 2022
(86) PCT No.: PCT/EP2022/053779

§ 371 Date: Aug. 15, 2023

(30) Foreign Application Priority Data
Feb. 16, 2021 (CH) 00149/21

(51) Int. Cl. A61K 35/745; A61K 9/00; A61K 31/353; A61K 36/73

(52) U.S. Cl. CPC A61K 35/745; A61K 9/0056; A61K 31/353; A61K 36/73

(57) ABSTRACT

The present invention relates to a composition for use in the prevention of respiratory allergy, in particular allergic rhinitis, dust mite allergy and/or asthma. The invention also provides a kit comprising a combination of at least one polyphenol and at least one Bifidobacterium longum in one or more containers.

### FIELD OF THE INVENTION

The present invention relates to a composition for use in the prevention of respiratory allergy, in particular allergic rhinitis, dust mite allergy and/or asthma. The invention also provides a kit comprising a combination of at least one epicatechin and at least one Bifidobacterium longum in one or more containers.

### BACKGROUND OF THE INVENTION

The incidence of allergic disorders has increased dramatically in the developed world and a variety of factors (e.g. environment, genetics, hygiene, diet) are known to impact on their development. Allergies may start in predisposed individuals from early infancy, e.g. as atopic eczema, limited to or followed by the development of food allergies. Respiratory allergies such as allergic rhinitis and asthma occur later in life and complete the sequel of allergic manifestations.

Recent advances in the understanding of the pathophysiology of allergy have revealed a series of complex immune cell interactions that could be manipulated to influence either sensitization to the allergen (i.e. prevention) or help to alleviate allergic manifestations and symptoms.

A majority of therapeutic options available today for respiratory allergies such as corticosteroids and anti-histaminics are aimed at treating different allergic symptoms. While proven to be highly effective, numerous factors such as dose, route and scheme of administration account for the efficacy of these treatments, notwithstanding the adverse effects that are sometimes associated with a chronic use of these medications.

Nutritional interventions to prevent the development or to manage the symptoms of allergic diseases are a promising alternative to medications and have been assessed in numerous pre-clinical and clinical settings. Typically, the protective effects of probiotic bacteria (Kalliomaki et al., Lancet. 2001, 357:1076-9.), prebiotics (Arslanoglu et al. 2008, J Nutr 138:1091-1095) and poly-unsaturated fatty acids (Dunstan et al. 2003, J Allergy Clin Immunol 112: 1178-1184) have been documented.

Dietary polyphenols and polyphenol rich plant materials have also been studied extensively in numerous disease models and published data suggest that their presence may have an important role in preventing tissue damage due to oxidative stress and hence might have an anti-allergic potential. Moreover, EP 2747584 from the same applicant, relates to epicatechin for use in the prevention or reduction of symptoms of an allergic disorder and/or an allergic reaction.

Also, probiotic strains have been described to play an important role in the gut microbiota and to be associated with positive health effects. They can promote good digestion, boost the immune system, and produce lactic and acetic acid that controls intestinal pH. Various probiotic cultures or mixes of probiotics have been described for their effect on the allergic immune system: for example, EP185833, describes a mix of probiotics that can decrease the risk of allergies due to wheat flour albumin and globulins. WO 2009/072889 describes a Bifidobacterium that can be used in improving the lung function of subjects suffering from dust mite allergy.

To a certain extent specific strains of B. longum have been described to have an antiallergenic effect. For example, patients suffering from allergic rhinitis due to Japanese cedar pollinosis showed reduced allergic symptoms after consumption of B. longum BB536 for several weeks before and during the pollen season (Xiao J Z et al. Effect of probiotic Bifidobacterium longum BB536 in relieving clinical symptoms and modulating plasma cytokine levels of Japanese cedar pollinosis during the pollen season. A randomized double-blind, placebo-controlled trial. J Investig Allergol Clin Immunol. 2006; 16(2):86-93.).

However, there still remains a need to find new and alternative compounds to prevent and reduce the symptoms of an allergic disorder or allergic reaction, without some of the inconveniences as mentioned above and/or for complementing the already existing choice of active ingredients. Particularly, the new solution should not have adverse health or side-effects upon a prolonged consumption, and the compound should ideally be available from a natural source and in amounts, which would make it practical and feasible to be consumed by an individual for example as part of a daily diet in a high enough dose for being effective.

### SUMMARY OF THE INVENTION

The object of the present invention is to improve the state of the art and to provide a new and alternative solution for preventing and/or reducing the symptoms of respiratory allergy disorder or related allergic reaction.

The object of the present invention is achieved by the subject matter of the independent claims. The dependent claims further develop the idea of the present invention.

Accordingly, the present invention provides in a first aspect at least one Bifidobacterium longum and at least one polyphenol for use in treating and/or preventing and/or alleviating respiratory allergy symptoms and/or a respiratory allergy reaction in an individual.

In a second aspect, the present invention provides a kit comprising a combination of at least one polyphenol and at least one Bifidobacterium longum in one or more containers.

It is one advantage of the present invention that it allows treating or preventing symptoms of a respiratory allergy disorder and/or an allergic reaction early.

It is another advantage of the present invention to decreases ocular symptoms, sneezing, sleep disorders thereby improving quality of life in allergic rhinitis sufferers.

It is also another advantage to further improve the depressive symptoms associated with such disorders.

### DETAILED DESCRIPTION OF THE INVENTION

### Definitions

Some definitions are provided hereafter. Nevertheless, definitions may be located in the “Embodiments” section below, and the above header “Definitions” does not mean that such disclosures in the “Embodiments” section are not definitions.

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a metabolite” or “the metabolite” includes one metabolite but also two or more metabolites.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions disclosed herein may lack any element that is not specifically disclosed herein. Thus, a disclosure of an embodiment using the term “comprising” includes a disclosure of embodiments “consisting essentially of” and “consisting of” the components identified.

The term “and/or” used in the context of “X and/or Y” should be interpreted as “X,” or “Y,” or “X and Y.” Similarly, “at least one of X or Y” should be interpreted as “X,” or “Y,” or “both X and Y.”

Where used herein, the terms “example” and “such as,” particularly when followed by a listing of terms, are merely exemplary and illustrative and should not be deemed to be exclusive or comprehensive. As used herein, “associated with” and “linked with” mean occurring concurrently, preferably means caused by the same underlying condition, and most preferably means that one of the identified conditions is caused by the other identified condition.

The terms “food,” “food product” and “food composition” mean a product or composition that is intended for ingestion by an individual such as a human and provides at least one nutrient to the individual. The compositions of the present disclosure, including the many embodiments described herein, can comprise, consist of, or consist essentially of the elements disclosed herein, as well as any additional or optional ingredients, components, or elements described herein or otherwise useful in a diet.

As used herein, the terms “treat” and “treatment” mean to administer a composition as disclosed herein to a subject having a condition in order to lessen, reduce or improve at least one symptom associated with the condition and/or to slow down, reduce or block the progression of the condition. The terms “treatment” and “treat” include both prophylactic or preventive treatment (that prevent and/or slow the development or progression of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional.

Both human and veterinary treatments are within the scope of the present disclosure. Preferably the at least one polyphenol and at least one Bifidobacterium are oleuadministered in a serving or unit dosage form that provides a therapeutically effective or prophylactically effective amount.

The terms “prevent” and “prevention” mean to administer a composition as disclosed herein to a subject is not showing any symptoms of the condition to reduce or prevent development of at least one symptom associated with the condition. Furthermore, “prevention” includes reduction of risk, incidence and/or severity of a condition or disorder.

As used herein, an “effective amount” is an amount that treats or prevents a deficiency, treats or prevents a disease or medical condition in an individual, or, more generally, reduces symptoms, manages progression of the disease, or provides a nutritional, physiological, or medical benefit to the individual.

The term “pet” means any animal which could benefit from or enjoy the compositions provided by the present disclosure. For example, the pet can be an avian, bovine, canine, equine, feline, hircine, lupine, murine, ovine, or porcine animal, but the pet can be any suitable animal. The term “companion animal” means a dog or a cat.

An “oral nutrition supplement” is a composition comprising at least one macronutrient and/or at least one micronutrient, for example in a form of sterile liquids, semi-solids or powders, and intended to supplement other nutritional intake such as that from food. Non-limiting examples of commercially available ONS products include MERITENE®, BOOST®, NUTREN® and SUSTAGEN®. In some embodiments, an ONS can be a beverage in liquid form that can be consumed without further addition of liquid, for example an amount of the liquid that is one serving of the composition.

A “kit” means that the components of the kit are physically associated in or with one or more containers and considered a unit for manufacture, distribution, sale, or use. Containers include, but are not limited to, bags, boxes, cartons, bottles, packages of any type or design or material, over-wrap, shrink-wrap, affixed components (e.g., stapled, adhered, or the like), or combinations thereof.

The term “allergic rhinitis”, also commonly known as pollenosis or hay fever, refers herein to an allergic inflammation of the nasal airways and characterized by blocked and runny nose, sneezing and itching. Ocular symptoms such as red and watery eyes can also manifest. It occurs when an allergen, such as pollen or dust mite, is inhaled by an individual with a sensitized immune system. In such individuals, the allergen triggers the production of the antibody immunoglobulin E (IgE).

The term “asthma” refers herein to the common chronic inflammatory disease of the lower airways and characterized by wheezing, mucus production and impaired lung function.

“Plant polyphenols” in the sense of the invention are phenols comprising more than 2 phenolic groups.

### EMBODIMENTS

The present invention pertains to a composition comprising at least one Bifidobacterium longum and at least one polyphenol for use in treating and/or preventing and/or alleviating respiratory allergy symptoms and/or a respiratory allergy reaction in an individual.

Examples of specific allergic disorders may be allergic rhinitis, dust mite allergy or asthma. The composition of the present invention may thus be for use in the treatment or prevention of an allergic disorder, wherein the allergen trigger is an environmental allergen, preferably selected from one or more of: dust mite, pollen, molds or mold spores, weed pollen, tree pollen, grass pollen, fleas, pet hair, feathers or pet dander.

The subject to be treated is preferably a mammal, preferably a human or a companion animal (pet), preferably wherein the subject is a child, an infant, an adolescent or an adult human, a dog, a puppy, a cat or a kitten. In a preferred embodiment, the individual is a person suffering from the pollen allergy.

The composition can be administered to a human being at all ages. However, advantageously, administration of the composition to a patient starts as soon as an allergy and its symptoms starts to manifest, i.e. in many instances already early in life. Hence, preferably, the human being is a young child between the age of 4 months and six years or an older child up to the age of 18 years or an adult person.

In an alternative embodiment, the composition is intended for consumption by an animal, preferably a cat or a dog. Similarly, as with humans, allergies and symptoms of such allergies can be observed with animals, in particular with domesticated animals and animals kept as pets. Advantageously, the current invention provides a solution which can be provided to a companion animal by his owner.

In an embodiment, the composition is effective for preventing and/or alleviating symptoms of respiratory allergy. Such symptoms may be sneezing, suffy nose, asthma, fatigue or tiredness, anxiety, sleep disorder. Symptoms associated with respiratory allergy are also mood swing/mood disorder, anxiety and/or depression. The present composition may thus further provide beneficial effects on such associated disorders.

The composition according to the present invention further supports immune system, in particular before and during pollen season.

### Composition

The composition according to the present invention comprises at least one polyphenol.

Polyphenols are a large group of small compounds with anti-oxidant activity that are commonly found in plants and fruits. Flavonoids are a sub-group of such polyphenols with shared structural features that themselves can be further sub-grouped for examples into flavonols, flavanols, flavones, flavanones and others. The plant phenols can be comprised in plant extracts. Thus, the invention also relates to a composition comprising plant extracts containing such polyphenols. Apples are rich in various polyphenols, including quercetin and phlorizidin; green tea is rich in different flavanols including procyanidins and gallated derivatives of catechins and epicatechins.

The composition can comprise at least one, at least two, at least three, or at least four different polyphenols. Non-limiting examples of natural sources of polyphenols can be apple, thyme, pomegranate, berries, green coffee, or mixtures thereof. The polyphenols may be extracted from natural plant sources by any known extraction technique, like an extraction with water or an organic solvent, like ethanol or ether.

In a preferred embodiment, the polyphenol is an epicatechin. Plant or fruit extracts rich in epicatechin or extracts supplemented with epicatechin or epicatechin in isolated or synthesized form per se, can be used. No undesired side-effects e.g. resulting from a strong corticosteroid or anti-histaminic activity are expected from a moderate use of epicatechin. Furthermore, plant or fruit extracts can be prepared which are on one hand effective for reducing allergy symptoms and on the other hand still pleasant for consumption. It is now also possible to provide extracts with the appropriate concentration of the active ingredient epicatechin that can be consumed in reasonable and feasible amounts per day e.g. as part of a daily diet by a consumer.

In one of the embodiments, the epicatechin is provided in the form of a plant extract or concentrate. This allows providing the epicatechin in its natural form and environment, but in concentrated condition. Thereby, the epicatechin is of natural origin and can be provided in a food product which is well recognized and appreciated by a consumer and still provide him with the necessary amount of active epicatechin. A further advantage is that the epicatechin does not need to be first produced in purified form, e.g. via chemical synthesis, and hence provides also a more economic solution for providing the epicatechin to a consumer.

Advantageously, the epicatechin is provided in the form of a plant extract which naturally already contains substantial levels of epicatechin and which are well liked by the consumers. Thereby, the plant extract or concentrate can preferably be an extract or concentrate from apple, cocoa or tea. In a most preferred embodiment, the extract is from apple.

In a preferred embodiment, the epicatechin is provided in the form of a composition comprising at least 0.1 wt %, preferably at least 1 wt % epicatechin. Alternatively, the epicatechin is provided in the form of a composition comprising at least 10 wt %, for example at least 20 wt % or 40 wt %. Thereby, the composition can be enriched with epicatechin either by concentrating the already naturally present epicatechin therein or supplementing the composition with for example a plant extract rich in epicatechin or with synthetically produced epicatechin. Advantageously, the resulting composition comprises epicatechin in such an amount that it is effective upon a normal, feasible daily consumption of said composition, for example if part of a food product. Alternatively, the composition which may be consumed between meals as a supplement and not as part of a regular meal.

The epicatechin is administered to a human being in an amount in the range from 25 mg to 10 g per day, from 50 mg to 10 g per day, preferably from 100 mg to 5 g per day, even more preferably from 300 mg to 1 g per day. These preferred doses allow to provide on one hand sufficient epicatechin to a relevant patient per day in order to provide the expected health benefit and on the other hand not to overdose epicatechin to prevent the risk of any potential undesirable or toxic effects to the patient.

The composition also contains at least one Bifidobacterium longum strain. In a preferred embodiment, the probiotic strain is Bifidobacterium longum ATCC BAA-999. The terms B. longum ATCC BAA-999 is intended to include the bacterium, parts of the bacterium and/or a growth medium fermented by the bacterium. The Bifidobacterium longum may be another available B. longum strain, such as B. longum NCC 2705 CNCM-I2618.

In one embodiment of the invention the composition comprises between 10⁴ and 10¹² colony forming units (CFU) per g of the dry composition. This amount may either be as a single dose, or spread across multiple doses. When the composition is an infant formula the amount of probiotic in the infant formula can be between 10⁵ and 10⁸ CFU/g of infant formula. In one embodiment the composition comprises between 10⁶ and 5×10⁷ CFU/g, that is in a dose demonstrated to have a physiological effect.

The probiotic strain can be mixed together with the dry or wet composition of the invention. Treatments or specific processes can be applied to improve the stability or viability of the probiotics in the composition. After mixing the probiotic with the composition the mix can be processed in a way that does not dramatically affect the viability of the probiotics. In another embodiment the probiotics are partly or fully inactivated before, during or after the mixing. In one embodiment the probiotics have been rendered inactivated and/or incapable to replicate prior to the use in the composition of the invention. This for example can be achieved by heat treatment or other known treatments in the art.

### Other Ingredients

The composition may further comprise Vitamin C, antioxidants and/or other bioactive ingredients. Vitamin C may be provided as natural source, e.g. from acerola extract. The amount provided daily depends on the age of the individual. Average daily recommended amount for adult is between 75 to 90 mg. The composition may provide at least 15% of the daily intake, up to 120% of the daily intake. Preferably, it provides 15 to 100% of the daily intake.

In a preferred embodiment, the composition comprises a mixture of B. longum ATCC BAA-999, epicatechin from apple extract and Vitamin C.

Further ingredients such as fruit extract may advantageously be added to the composition. Examples of such fruit may be goji berry, mulberry, orange.

### Format

The composition for use of the invention is provided in the form of a composition, which is a food product, a food supplement or a pharmaceutical product.

Typically, the composition may be selected from the group consisting of a food composition, a pet food composition, a dietary supplement, a nutraceutical, a nutritional formula, a drink, and/or a medical composition. Examples of food compositions that are applicable to the present invention are yoghurts, milk, flavored milk, ice cream, ready to eat desserts, powders for re-constitution with, e.g., milk or water, chocolate milk drinks, malt drinks, ready-to-eat dishes, instant dishes or drinks for humans or food compositions representing a complete or a partial diet intended for pets or livestock. Consequently, in one embodiment the composition according to the present invention is a food product intended for humans, pets or livestock, and preferably humans and pets. In a preferred embodiment, the composition is a food product, or a dietary supplement intended for humans (infant, child, adolescent, or adult) or companion animals (pets) (preferably dog, puppy, cat or kitten).

A particular need for products to reduce symptoms of respiratory allergies may be in the clinical environment. Therefore, in a still further embodiment, the food product is a food for specific medical purposes such as a health care food product for oral feeding, and/or a nutritional product for enteral feeding. The advantage of the invention is that the active ingredient polyphenol (preferably epicatechin) can be provided in relatively high local concentration and low volumes of a medical food product and hence be administered effectively to patients in such need.

In another embodiment, the food product is selected from the group consisting of an infant feeding composition, a follow-up formula, a growing-up milk, an infant cereal, or a baby food product. These products are particularly well suited to address and solve the problem of the prevention or reduction of symptoms of allergies in babies and young children.

In a further embodiment, the food product is selected from the group consisting of a beverage product, a yoghurt product, a fermented milk, a fruit juice, or a cereal bar. These food products are well suited for administering the composition to older children and adult humans. The food products can well be enriched with polyphenol (preferably epicatechin) and B. longum and have a credible image to provide a health oriented functional food product to the consumers.

In a still further embodiment, the food product is a pet food product.

The pharmaceutical or veterinary formulation may be in the form of a tablet, a capsule, granules, or a powder. The composition may be a shelf stable powder. The low water activity provides this shelf stability and ensures that probiotic micro-organisms, will remain viable even after long storage times.

It can be in a single serving or dose of the composition comprising the effective amount of the active ingredients or in a separate composition for simultaneous or sequential administration. A package can contain one or more of the servings or doses.

The compositions are administered in an amount sufficient to at least partially treat or arrest the symptoms of the allergic disorder. An amount adequate to accomplish this is defined as “a therapeutically effective dose”. Amounts effective for this purpose will depend on a number of factors known to those of skill in the art such as the severity of the disease and the weight and general state of the patient.

In prophylactic applications, compositions according to the invention are administered to a patient susceptible to or otherwise at risk of a particular disease in an amount that is sufficient to at least partially reduce the risk of developing a disease. Such an amount is defined to be “a prophylactic effective dose”. Again, the precise amounts depend on a number of patient specific factors such as the patient's state of health and weight. Generally, epicatechin and B. longum ATCC BAA-999, will each be administered in a therapeutically effective dose and/or in a prophylactic effective dose.

In some embodiment, the composition is administered at least once a day. In the management of pollen allergy, the composition is preferably administered orally at least 4 weeks before the start of the pollen season.

### Kit for Use

A kit comprising a combination of at least one polyphenol and at least one B. longum in one or more containers.

In an embodiment of the kit, the one or more containers comprise at least one first container that stores at least one polyphenol separately from at least one B. longum strain, which is stored in at least one second container, and the kit further comprises instructions for admixing the polyphenol with the B. longum strain into a unit dosage form.

In a preferred embodiment, the polyphenol is epicatechin, preferably in the form of an apple extract.

In a preferred embodiment, the B. longum strain is ATCC BA-999.

In an embodiment of the kit, the combination can be provided together in one or more prepackaged unit dosage forms, for example in separate containers that each contain a dried powder such that each container contains one prepackaged unit dosage form.

In another embodiment, the kit can comprise a plurality of compositions for admixing together to form one or more of the compositions disclosed herein. For example, the kit can contain two or more dried powders in separate containers relative to each other, the separate powders each containing a portion of the final unit dosage form. As a non-limiting example of such an embodiment, the kit can contain one or more first containers that house the at least one epicatechin and can also contain one or more second containers that house the B. longum. The content of one of the first containers can be admixed with one of the second containers to form at least a portion of the unit dosage form of the composition.

In a preferred embodiment, the composition is packed in individual primary packages. The primary package can be a blister pack, a rupturable capsule, a flask, a bottle, a breakable glass ampoule, or any other similar packaging for containing and dispensing one dose of the composition. In one more preferred embodiment, the primary package is an individual sachet made of a flexible material. The material is preferably barrier to oxygen and moisture to guarantee a shelf-life of at least 6 months, preferably at least 12 months. The material can be made of a monolayer or multilayer film, with or without a metallic layer, selected within the list of the following materials: cellulose fibers, paper, cardboard, polyolefins, biodegradable polymers such as polylactic acid (PLA), polybutylene succinate (PBS), polybutylene adipate terephthalate (PBAT), polyhydroxy alcanoate (PHA), or a combination thereof. If the material is a multilayer, it can further contain adhesive layers, primers, protective layers, inks.

In an alternative embodiment, the primary package may contain other bioactive ingredient than those described above, i.e. any other functional ingredient known in the art.

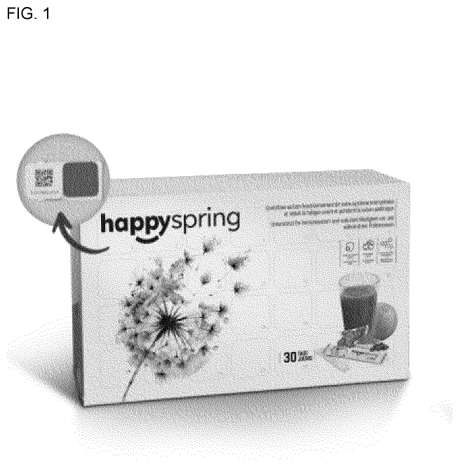

Each primary package is then contained in a secondary packaging. In one preferred embodiment, the secondary packaging is a carton box comprising a plurality of individual compartments disposed adjacent to one another, as illustrated in FIG. 1, each for containing a primary package. Each compartment is closed by a wall that is opened manually by the consumer at the time of consumption of the composition. The wall preferably comprises precuts to detach said wall at least partly. In a more preferred embodiment, the wall is detachable over three of its sides, so that when detached along the precuts, the fourth side acts as a hinge to create a door-like panel. This prevents packaging material to be fully detached from the rest of the secondary packaging, hence ensuring proper disposal and recyclability of the entire packaging.

In another embodiment, the composition may be different from one compartment to the other, in terms of dosage, but also in terms of bioactive ingredients that are present into the composition. Such bioactive ingredients are preferably the one described above but may alternatively be any other functional ingredient.

In a particularly preferred embodiment of the invention, each compartment comprises an information associated to the composition. That information is preferably printed as a QR code or similar type of code. The code is adapted to be read by a sensing device, for instance the camera of a portable communication device that can then display information retrieved from a remote database. The information can be identical from one compartment to another, but advantageously can be different from one compartment to another, and adapted to each individual intake of the composition. The information (e.g. the QR code) is preferably printed at the inner surface of the detachable wall of each compartment, such that when the consumer opens one compartment, the information can be delivered at the same time as the composition intake.

The QR code may also provide educational information, link to website and shop. In some embodiment the website allows to share feedback.

In a highly preferred embodiment of the invention, the secondary packaging comprises as many compartments as days in a month. The number of compartments can vary from one secondary packaging to another, depending on when it is to be used during the year. This provides the consumer with a way of consuming one dose of the composition, each day, and also benefit from a dedicated information, also for each day of the month. This is particularly advantageous for prevent under-, or over-intakes of the composition, ensuring regularity of intakes throughout the month (monitor daily consumption).

Those skilled in the art will understand that they can freely combine all features of the present invention disclosed herein. In particular, features described for different embodiments of the present invention may be combined. Further advantages and features of the present invention are apparent from the figures and examples.

### Example 1: Food Supplement

The composition is provided as a 2 stick packs to be reconstituted in water.

The first 8 g stick pack comprises the epicatechin along with mixture of fruit extracts (see Table 1). The second stick pack only contains as active ingredient the probiotic bacteria strain Bifidobacterium longum ATCC BAA-999 in a maltodextrin powder matrix (33% B. longum strain, 67% maltodextrin). The B. longum strain is in an amount of 7.25E10 CFU/g (0.75 g total).

TABLE 1

| Recipe Stick 1 | |
|---|---|
| Apple extract polyphenol | 0.2 |
| Acerolife | 0.3 |
| Goji (Fruits) | 0.3 |
| Raspberry | 2.5 |
| Blood Orange | 4.7 |
| Total | 8 |

Both stick packs are to be mixed together before addition of 100 ml water. The reconstituted mix is then immediately consumed by the individual.

1. A method for use in treating and/or preventing and/or alleviating respiratory allergy symptoms and/or a respiratory allergy reaction in an individual comprising administering a composition comprising at least one of polyphenol and at least one of Bifidobacterium longum to an individual in need of same.

2. Method for use according to claim 1, wherein the respiratory allergy is selected from the group consisting of allergic rhinitis, dust mite allergy and asthma.

3. Method for use according to claim 1, wherein the symptoms are selected from the group consisting of ocular symptoms, sneezing, stuffy nose, asthma, fatigue and/or tiredness, anxiety, sleep disorder, mood disorder, and depression.

4. Method according to claim 1, wherein the composition further reduces anxiety, swing/mood disorder and/or depression associated with respiratory allergy.

5. Method according to claim 1, wherein the composition further supports immune system before and during pollen season.

6. Method according to claim 1, wherein the individual is a person suffering from pollen allergy.

7. Method according to claim 1, further comprising Vitamin C.

8. (canceled)

9. Method according to claim 1, wherein the Bifidobacterium longum is ATCC BAA-999.

10. Method according to claim 1, wherein the polyphenol is an epicatechin.

11. Method according to claim 1, wherein the polyphenol is provided in the form of a plant extract or concentrate.

12. Method according to claim 1, wherein the epicatechin is provided in the form of a composition comprising at least 0.1 wt % epicatechin.

13. Method according to claim 1, wherein the epicatechin is administered to the individual in an amount in the range from 25 mg to 10 g per day.

14. Method according to claim 1, wherein the composition is in a form selected from the group consisting of a food product, a pet food product, a food supplement and a pharmaceutical product.

15. Method according to claim 1, wherein the at least one Bifidobacterium and the at least one polyphenol are provided together in one or more prepackaged unit dosage forms, or separately in one or more prepackaged unit dosage forms.

16-22. (canceled)
